(19) United States
(12) Patent Application Publication
(10) Pub. No.: US 2024/0024523 A1
(43) Pub. Date: Jan. 25, 2024

(54) SANITIZATION APPARATUS AND DOORKNOB

(71) Applicant: Dai ARAI, Tokyo (JP)

(72) Inventor: Dai ARAI, Tokyo (JP)

(21) Appl. No.: 18/480,485

(22) Filed: Oct. 3, 2023

Related U.S. Application Data
(63) Continuation of application No. PCT/JP2022/012930, filed on Mar. 21, 2022.

(30) Foreign Application Priority Data
Apr. 5, 2021 (JP) 2021-064482

(51) Int. Cl. A61L 2/04; A61L 2/24; E05B 1/00

(52) U.S. Cl. CPC A61L 2/04; A61L 2/24; E05B 1/0069; A61L 2202/14; A61L 2202/17

(57) ABSTRACT

Provided is a sanitization apparatus including a rotation drive portion, an annular thin film which has a predetermined perimeter and covers an outer circumference of the rotation drive portion, and a fixed base portion to which the rotation drive portion is attached, in which the rotation drive portion has a disinfection portion which disinfects the thin film, and the fixed base portion has a drive shaft which causes the thin film to perform rotational movement along the outer circumference of the rotation drive portion to cause the thin film to sequentially pass through the disinfection portion.

The contents of the following patent application(s) are incorporated herein by reference:
No. 2021-064482 filed in JP on Apr. 5, 2021, and
No. PCT/JP2022/012930 filed in WO on Mar. 21, 2022.

### BACKGROUND

### 1. Technical Field

The present invention relates to an apparatus which inactivates or reduces a pathogen (such as a bacterium or a virus) adhered to an object surface.

### 2. Related Art

Even in Japan, which is relatively in good hygienic conditions as compared to foreign countries, there is no end to infectious diseases due to a seasonal flu or a norovirus every year. In addition, during recent years, outbreaks of a new flu, a new coronavirus, and the like also became new threats to humans. Although their infection routes are diverse, contact infection can be mentioned as one of common infection routes. That is, a route of indirect contact infection resulting from a pathogen on a hand or the like of a carrier adhering to an object surface due to a touch to a doorknob, a handrail, or the like by the carrier, followed by a touch by a healthy individual.

Up to now, such a doorknob that is to rapidly inactivate or reduce a pathogen on the object surface to which a pathogen is likely to frequently adhere has not been proposed.

### LIST OF CITED REFERENCES

### Non-Patent Document

Non-Patent Document 1: “Viruses Contained in Droplets Applied on Warmed Surface Are Rapidly Inactivated”, Fiquet S. et al., 2014

An issue to be solved is to inactivate or reduce a pathogen adhered to an object surface, and achieve a cleaned state of the object surface by the time of next use of the object.

### SUMMARY

To solve the above described challenge, the present invention proposes an apparatus as follows.

An object surface desired to be used in a cleaned state is temporarily retreated from a position of use, and a heating treatment is performed in the retreat position. Heating conditions (such as a temperature and a heating time) at this time are optional depending on a type of a pathogen desired to be inactivated or reduced or a requirement on how much the pathogen is desired to be reduced.

The meaning of retreating the object surface also includes a configuration of covering the object surface with a cover or the like during heating.

Regarding the heating of the object surface, generally, the higher the heating temperature, the shorter the inactivation time of a pathogen. Thus, a higher heating temperature is desired. On the other hand, in most cases, use is not possible if the temperature at the time of use is such high temperature. For example, although most pathogens are killed or inactivated if the doorknob is heated with 200 degrees Celsius for 10 seconds, a person cannot touch the doorknob until it returns to a room temperature.

Thus, the present invention is focused on keeping a heat capacity of an object surface subjected to heating disinfection to the minimum necessary. That is, the minimum necessary heat capacity suggests heating a thin material such as a film or a thin plate. An apparatus is to include means to heat only this thin material for realization of desired inactivation or reduction, and to place this thin material on a base material or framework which has a required rigidity.

In accordance with this apparatus, since the thin material which provides disinfection has only the minimum necessary heat capacity, even when a human hand touches the thin material immediately after it is heated to a high temperature, there is no risk of a burn injury. In addition, even when the thin material is cooled after the high temperature heating, since the stored heat quantity is low, rapid heat dissipation (cooling) is carried out in the same way as in the case of the heating. Furthermore, since the thin material has a wide area contacting the external air, it rapidly returns to a room temperature only with exposure to the air of a room temperature after the heating. Accordingly, by selecting a material having an appropriate specific heat and thickness, and by setting a heating temperature and a cooling time until a person touches the heated surface, there is no risk of a person suffering from a burn injury after the high temperature heating.

Furthermore, heating only the thin material means that energy put into the heating can be extremely little. For example, with a heat quantity for raising a temperature of water with a thickness of 100 μm by 9 degrees Celsius, it is possible to raise a temperature of a SUS 304 foil with a thickness of 10 μm by as high as 188 degrees Celsius. When a room temperature is 23 degrees Celsius, after the temperature rise by 188 degrees Celsius to reach 211 degrees Celsius, a pathogen can be made almost non-toxic within 10 seconds.

To paraphrase the previous paragraph, even when hands and fingers touch the SUS 304 foil with a thickness of 10 μm which has been heated to 211 degrees Celsius at the room temperature of 23 degrees Celsius, a burn injury does not occur. A reason for this is that a heat storage amount when a temperature of the SUS 304 foil with a thickness of 10 μm rises by 1 degree Celsius is only 0.05 times that of keratin of hands and fingers with a thickness of 100 μm, and even when the heat storage amount of the SUS foil at 211 degrees Celsius is distributed between the SUS foil and keratin until the temperature becomes the same, the temperature only reaches 45 degree Celsius. This means that a person can only feel like holding a teacup filled with tepid water.

In this manner, at the time of performing heating disinfection on a surface of a structural body, it is extremely effective means in terms of disinfection efficiency and also in terms of energy saving to separate the surface as thin as possible and perform heating disinfection only on the separated surface with a high temperature.

The heating temperature and time will now be described. When qualitatively considered, there is no doubt that the longer hours to a maximum extent the heating is performed with the higher temperature to a maximum extent, inactivation/reduction of a pathogen can be expected. However, in the actual product design, setting and designing should be done in accordance with a requirement specification of the apparatus in consideration of efficiency and economics. There is one rough standard that heating should be performed to a boiling point of water or higher if sterilization is intended. Bacteria are microorganisms unlike viruses, and moisture is stored within a cell wall. Accordingly, if a bacterium is heated to the boiling point or higher, at the instant the water is vaporized, swelling occurs and the cell wall is destroyed due to internal pressure, and the bacterium can no longer survive. Therefore, if heating is performed to the boiling point of water or higher, that is, 100 degrees Celsius or higher in the atmosphere, and adhered bacteria can be brought to a state of 100 degrees Celsius or higher, the bacteria can be instantly killed.

On the other hand, this does not necessarily apply to viruses. Since viruses and phages are merely chemical substances and not organisms, their molecular structures may be maintained for a while even in an environment at 100 degrees Celsius or higher. Therefore, the heating temperature and the heating time may be decided in view of an allowable temperature limit of a metal foil or a resin film to be used, a temperature of a heating body, economics, and safety.

Specific examples thereof will be described. Viruses include a virus with a coating called an envelope and a virus without an envelope. For example, flu viruses and coronaviruses are types having envelopes, and noroviruses are types not having envelopes. Although intuitively those having coatings seem to have more external resistance, actually they do not. This is because the envelope types can be inactivated by only rupturing its envelopes. On the other hand, the non-envelope types cannot be inactivated unless its structure itself is destroyed. Accordingly, regarding the envelope types, it can be expected that moisture inside an envelope will be vaporized and the envelope will be destroyed from the inside by heating to the boiling point of water or higher, as in the case of heating disinfection of bacteria.

Here, a scientific paper which supports the study will be cited. In Non-Patent Document 1, experiments are conducted to actually measure a degree of reduction of infectivity when high temperature heating is performed on an enveloped virus and a nonenveloped virus along with a heating time. According to this, when the enveloped virus is heated at 100 degrees Celsius, the infectivity can be reduced to 1/10,000 of its original infectivity in one second (at a detection limit or below). The nonenveloped virus on the other hand requires nine seconds at 100 degrees Celsius. When the nonenveloped virus is heated at 130 degrees Celsius, the infectivity can be reduced to the detection limit in two seconds.

In this manner, since the heating conditions (the temperature and the time) are different depending on which pathogen is to be reduced and how much, the present invention cannot be uniformly defined. However, when immediate effect and effectiveness are taken into account, heating to 100 degrees Celsius (boiling point of water) at which a bacterium and an enveloped virus are instantly killed and inactivated or above is considered as a rough standard for a lower limit temperature. On the other hand, examples of a restriction on an upper limit side include that a person does not suffer from a burn injury when touching in a heat storage state at the temperature (depending on a thickness, a specific heat, and a head conduction of the material), that a thin film material does not significantly degrade even after rise to the temperature is repeated, that a period of time to rise to the temperature is not significantly long, and that a voltage and a current until the rise to the temperature is completed is allowed from a viewpoint of safety and economics. There are not so many materials satisfying these conditions at the moment, but those will be described in embodiments.

### EFFECT OF THE INVENTION

According to the present invention, a surface of an object that requires cleaning can be rapidly and automatically disinfected or attenuated in an energy saving way. With this configuration, it is possible to respond to social needs for demanding that a doorknob, a handrail, or the like where an object surface is frequently contaminated is to be cleaned each time the contamination occurs.

### BRIEF DESCRIPTION OF THE DRAWINGS

FIG. 1A is a representative drawing of the present invention, illustrating an apparatus appearance diagram.

FIG. 1B is a representative drawing of the present invention, illustrating a central cross-sectional view of a sanitization apparatus 101.

FIG. 2 is an outer appearance view of an apparatus of the present invention in which a cover part is half cut.

FIG. 3 is a state diagram at the time of maintenance of the apparatus of the present invention.

FIG. 4 is a state diagram at the time of maintenance of the apparatus of the present invention (diagram as viewed from an opposite side of FIG. 3).

FIG. 5 is a state diagram at the time of maintenance of the apparatus of the present invention.

FIG. 6 is a cross-sectional view of a base apparatus part of the apparatus of the present invention.

FIG. 7A is a cross-sectional view for describing a rotation drive portion.

FIG. 7B is an A-A cross-sectional view of the rotation drive portion.

FIG. 7C is an isometric view of the A-A cross section of the rotation drive portion.

FIG. 8 is a flowchart of software for driving the apparatus of the present invention.

### DESCRIPTION OF EXEMPLARY EMBODIMENTS

A core intent of the present invention is to perform heating disinfection on only a minimum surface thickness part of a functional structure desired to be cleaned (such as a doorknob or a handrail). However, if a part for which cleaning is desired is made too thin or too small, issues of rigidity and durability may occur. On the contrary, if the part is made thick to a certain level, there are disadvantages that frequent use cannot be withstood because long heating time and cooling time are required, and great energy is required.

Accordingly, the thickness and the structure of the apparatus are to meet heating conditions based on design requirements on how much a pathogen is to be reduced within what seconds and the apparatus then returns to an available state. Its design realization means is countless and is not uniformly determined. The following embodiments illustrate only one example using the present invention among those configurations that can withstand practical design.

### Embodiments

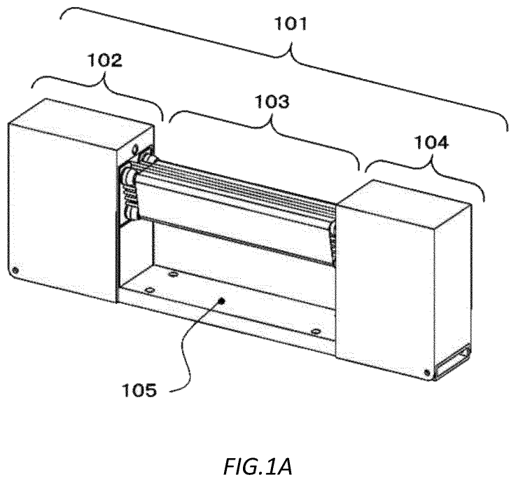

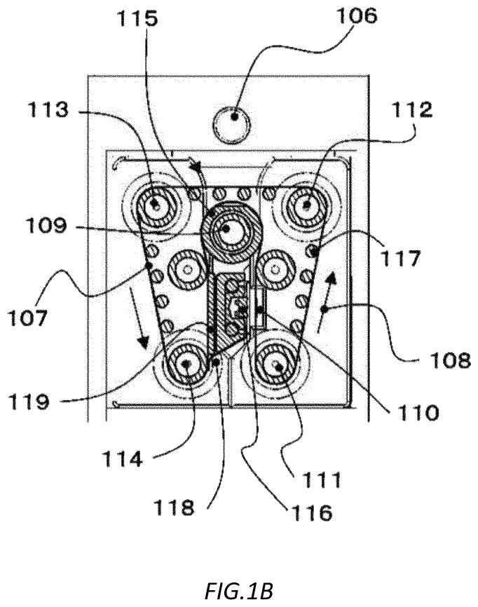

FIG. 1A illustrates an apparatus appearance diagram as an embodiment of the present invention, and FIG. 18 illustrates a central cross-sectional view. In FIG. 1A, by driving force obtained by rotation of a drive shaft 109, a thin film 107 travels in an orientation of an arrow 108 in a rotation direction. When the drive shaft 109 is regarded as a starting point, the thin film 107 passes through a heating disinfection band 110 to then exit to an apparatus surface side (outside), and sequentially passes through driven rotary shafts 111, 112, 113, and 114 to then enter into the inside of the apparatus again. The thin film 107 follows a route to have a surface of the thin film cleaned by a scraper 118 and a wiping sheet 119 for cleaning the thin film surface and to return to the drive shaft 109 again. The scraper 118 and the wiping sheet 119 are examples of a wiping portion which removes a water droplet or dirt adhered to the surface of the thin film 107.

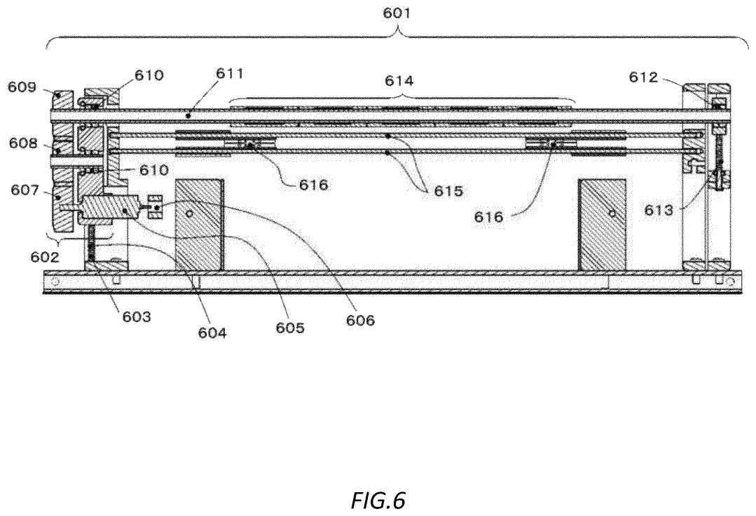

The drive shaft 109 is provided to a fixed base portion which will be described below, and causes the thin film 107 to perform rotational movement along an outer circumference of a rotation drive portion which will be described below, so that the thin film 107 is caused to sequentially pass through a disinfection portion. A thickness of the thin film 107 may be 20 μm or less. Since the drive shaft 109 needs to have a function of feeing out the thin film 107, the drive shaft 109 is integrated and provided with a friction resistive body 115 which has an appropriate friction coefficient on its outer circumference. The friction resistive body 115 causes the thin film 107 to perform rotational movement by frictional force against the thin film 107. In addition, to take up slack of the thin film 107 and to obtain appropriate frictional force between the thin film 107 and the friction resistive body 115, the drive shaft 109 generates tension by a spring in an upward direction in the drawing. This structure will be described below with reference to FIG. 6.

The thin film 107 is an annular thin film having a predetermined perimeter and covering the outer circumference of the rotation drive portion which will be described below. Since the thin film 107 is heated to 100 degrees Celsius or above by the heating disinfection band 110, the thin film 107 is required to have heat resistance to at least deal with the temperate. When economics and availability of the thin film are taken into account, the thin film 107 is desirably, for example, a polyimide or PTFE thin film. It can be mentioned that from a viewpoint of availability and a viewpoint of a required heating time, a thickness is desirably from 5 μm to 12.5 μm. The thinner the thin film is, the heating time shorter, and the thin film is desirable to be thinner in terms of input energy and a time required. However, a polyimide film with a thickness of, for example, 5 μm is fragile, and is likely to be ruptured. Depending on a required specification of a client, when durability and availability are mainly taken into account, a polyimide film with a thickness of 7.5 μm or 12.5 μm is good as for a balance of apparatus performance.

The heating disinfection band 110 is an example of the disinfection portion which is provided to the rotation drive portion and disinfects the thin film 107. The heating disinfection band 110 may heat the thin film 107 to 100 degrees Celsius or above. The heating disinfection band 110 generates heat by electrical resistance. Accordingly, any material may be used basically as long as the material is heated by energization. The heating disinfection band 110 may be, for example, a nichrome wire or an SUS plate. Experiments were conducted using several types of materials and shapes. When an SUS 304 was used as the material of the heating disinfection band 110, a width was set at 3 mm, a thickness was set at 0.01 mm, and a length was set at 160 mm, an entirety including conductive wires connected to both ends of a heating band was applied with 7.8 V, and 1.3 A flew therethrough.

At this time, when it is assumed that a volume resistance value of the SUS 304 is 93 μΩcm, a density is 7.93 g/cm\hat{ }3, a specific heat is 280 J/kg·degrees Celsius, the heating disinfection band 110 consumes 8.4 W. When it is assumed that heat dissipation and heat transfer do not occur, a calculation is that the temperature rises by 788 degrees Celsius in one second.

The thin film 107 is caused to travel while being in contact with this heating disinfection band 110. At this time, when it is assumed that the thin film is caused to travel to pass through the heating disinfection band 110 with a width of 3 mm in 0.2 seconds, from the calculation in the previous paragraph, the thin film receives energy for the rise of the heating disinfection band 110 by 158 degrees Celsius during this period. Not all the energy is necessarily transferred from the heating disinfection band 110 to the thin film 107, of course. In addition, although a situation is different depending on an ambient temperature (room temperature), as a rough standard, specification setting and design may be carried out while those levels of the voltage, the current, and the period of time to pass through are set as references.

Incidentally, at a timing at which the thin film 107 passes through this heating disinfection band 110, a thin film position sensor 116 senses a thin film position mark 705. The thin film position sensor 116 can sense rotational movement of the thin film 107 by sensing the thin film position mark 705. The thin film position mark 705 is a mark on a surface of the thin film 107, which is applied with heat resistant paint. The thin film position sensor 116 is a reflective photo reflector, and reads an increase or a decrease of reflected light from the thin film position mark 705. When a grip apparatus has the thin film position sensors 116 and the thin film position marks 705 equipped at both ends, it is possible to check a travel state of the thin film 107 in a manner of an A phase and a B phase of an encoder. Specifically, such functions can be provided that when a sensor does not respond within a certain period of time, since it is considered that the travelling of the thin film has difficulty due to some trouble or that the thin film is damaged or contaminated, the apparatus can be stopped and an error message can be issued, and a starting point during one rotation in a loop of the thin film 107 can be stably reproduced by determining, as a reset position, a position where a sensor turns ON together with the sensors at both ends of the grip.

Both ends of the driven rotary shafts 111 to 114 are supported by rotational bearing such that the thin film 107 can travel smoothly. In addition, although a fixed support structural body 117 does not rotate, if a frictional resistance is high, a smoothing treatment such as covering with a heat shrinkable fluoropolymer tube may be performed.

The scraper 118 and the wiping sheet 119 are placed to eliminate dirt and a water droplet adhered to the surface of the thin film 107. A purpose for this is that even when the thin film 107 passes through the heating disinfection band 110 in a state in which a large water droplet is still adhered to the thin film, a heat quantity enough for the entire water droplet to evaporate is not provided, and that is why a large, adhered object is eliminated in advance. The scraper 118 has a role as a wiper which scrapes the water droplet adhered to the surface, and an appropriate material is a thin film resin material such as polyimide film. In addition, a material of the wiping sheet 119 needs to have water absorption property. A common absorbent paper, non-woven fabric, or the like may be used, but the material desirably has elastically so that the thin film 107 is pressed against this wiping sheet 119 at certain force. Thus, by taking economics into account too, a felt material was adopted in a trial product, and a good result was obtained.

As above, a situation where the loop material of the thin film 107 goes around to be disinfected has been described. Next, the entire sanitization apparatus in which this structure is accommodated will be described.

A sanitization apparatus 101 of FIG. 18 will be described here. The sanitization apparatus 101 is broadly constituted by three types of portions including a base portion 105 fixed to a door, a doorknob, or the like, a gripping portion 103 which rotationally drives the thin film 107 to be disinfected, and fixing portions 102 and 104 which fix the gripping portion 103. Since the summary of the gripping portion has been described in the previous paragraphs, hereinafter, other parts will be mainly described. The sanitization apparatus 101 may include a human sensor 106 which senses use of the sanitization apparatus 101. When the human sensor 106 senses a person during the rotational movement of the thin film 107, the sanitization apparatus 101 may stop the rotational movement of the thin film 107. When the human sensor 106 does not sense a person during a predetermined period, the sanitization apparatus 101 may intermittently perform disinfection drive of the thin film 107. The sanitization apparatus 101 may be programmed to sense that disinfection is not able to be performed every two seconds.

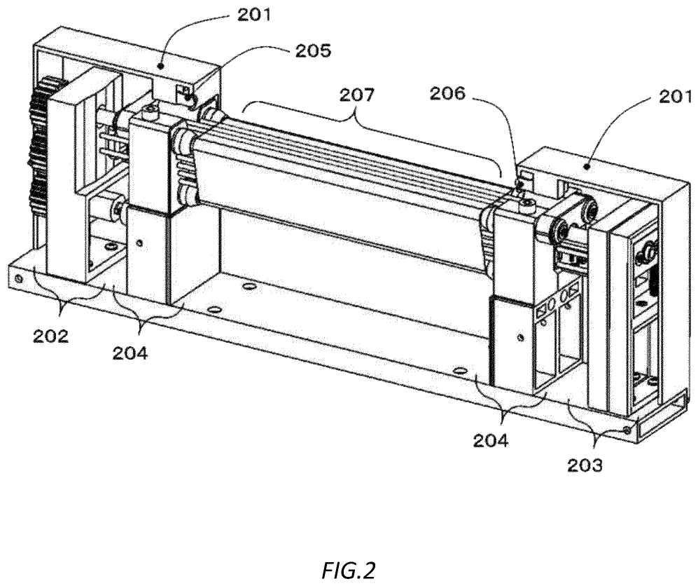

FIG. 2 illustrates a half cut view of covers 201 of the fixing portions 102 and 104. The inside of the sanitization apparatus is illustrated in the drawing. Note that to simplify the drawing, illustrations of various wires are omitted. This diagram is divided into a motor and gear wheel support portion 202, a central shaft support portion 203, and a rotation drive portion support portion 204. In addition, the human sensor separately includes a human sensor light source 205 and a human sensor light reception portion 206, and can sense that a human hand or the like approaches a grip portion 207. Note that in the present diagram, the human sensor is intended to be a photo interrupter, but can also be constituted by an ultrasonic sensor.

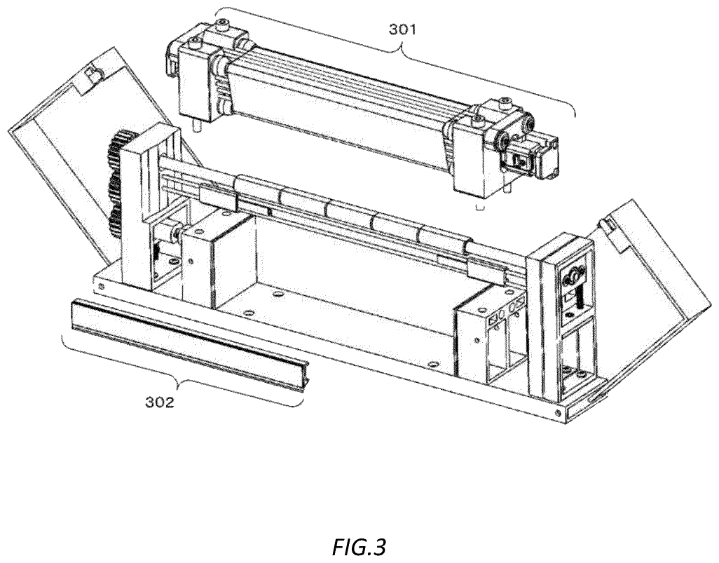

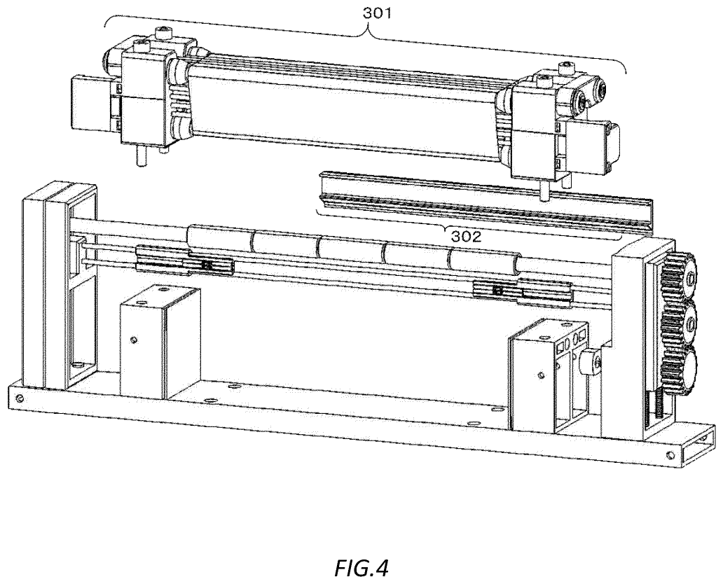

FIG. 3 is a maintenance diagram at the time of replacement of consumable parts. In preparation for a deformation due to an unexpected load, a rotation drive portion 301 itself is formed as a replaceable unit. In addition, a wiping portion 302 alone which has a scraper and a wiping sheet is detachable, and it is possible to easily replace the wiping portion when the contamination becomes severe. FIG. 4 also illustrates a diagram of this as viewed from an opposite side.

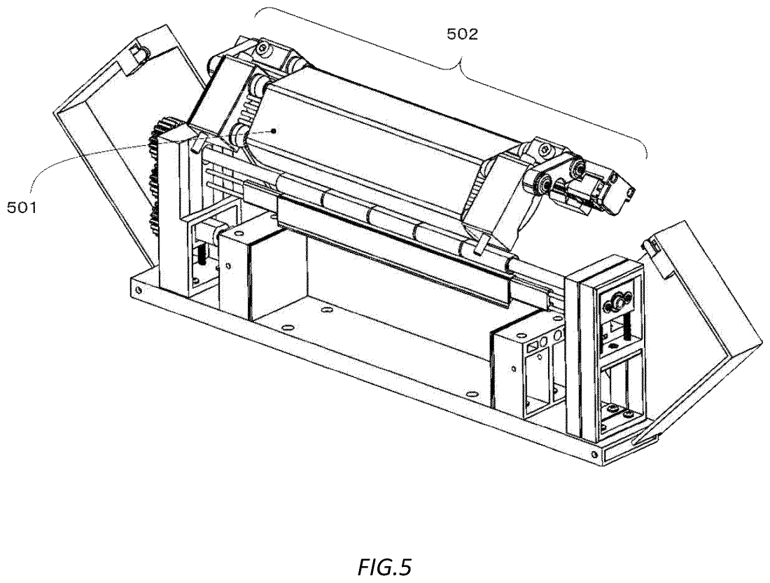

In addition, since a total perimeter of a loop of a thin film 501 is set to be longer than a total length of external dimensions of a rotation drive portion 502, a loop alone of the thin film 501 is also replaceable. This image is illustrated in FIG. 5. The rotation drive portion 502 is attached to the fixed base portion after the thin film 501 is attached to an outer circumference of the rotation drive portion. Note that when the rotation drive portion 502 is docked to a main body of the sanitization apparatus, since the rotation drive portion is nested with a drive shaft portion and the wiping portion 302, the rotation drive portion 502 has an opening and closing structure using a butterfly structure as illustrated in the drawing. According to the opening and closing structure of the rotation drive portion 502, opening and closing may be performed such that the perimeter of the rotation drive portion 502 varies. A perimeter in a state in which the rotation drive portion 502 is opened may be longer than a perimeter in a state in which the rotation drive portion 502 is closed. In a state in which the opening and closing structure is closed, a perimeter of the thin film 501 may be longer than a perimeter of a flange of the rotation drive portion 502. With this configuration, the thin film 501 can be attached to the rotation drive portion 502. In a state in which the opening and closing structure is opened, the perimeter of the thin film 501 may be longer than a perimeter of the outer circumference of the rotation drive portion 502 covered by the thin film 501. With this configuration, the rotation drive portion 502 can be attached to the fixed base in a state in which the thin film 501 covers the circumference of the rotation drive portion 502. Note that at the time of the operation of the sanitization apparatus 101, with regard to the rotation drive portion 502, in a state in which the opening and closing structure is closed, the drive shaft 109 may cause the thin film 107 to perform the rotational movement, and the disinfection portion may disinfect the thin film 501.

Then, to facilitate understanding of the apparatus structure, the apparatus is separated into a structure of each unit, and a description thereof will be provided below. First, FIG. 6 illustrates a cross-sectional view of a fixed base portion 601. The rotation drive portion may be attached to the fixed base portion 601. A motor and gear support portion 602 is supported in a state with a preload is applied which is upward in the drawing by a spring 603. Note that a movement direction is restricted to only a vertical direction by a guide shaft 604 which penetrates through the inside of the spring 603 and the motor and gear support portion 602.

A motor 605 has an encoder 606 mounted thereto, and can monitor a drive state by itself alone. That is, since it is possible to monitor an excessive load state or the like where the motor does not rotate even though a current is applied thereto, overheating can be avoided. A gear wheel 607 directly connected to a motor shaft transfers rotations to a gear wheel 609 via an intermediate gear wheel 608. Each gear wheel is supported by a rotational bearing 610 to smooth the rotations.

A gear wheel 610 is directly connected to a drive shaft 611, and serves as a power source for rotating a drive shaft 611. A support portion on an opposite side of the drive shaft 611 is also supported by a rotational bearing 612, and an upward preload is applied by a spring 613 to the drive shaft 611 via the rotational bearing in the same way as described above. That is, a structure is adopted where this force of the springs on both sides applies tension to the thin film 107 via the drive shaft to take up slack. The fixed base portion 601 may have a position control portion which controls a position of the drive shaft 611 to apply the tension to the thin film 107. The spring 603 and the spring 613 are examples of the position control portion.

The drive shaft 611 includes a friction resistive body 614 in a central section. A loop body of the thin film 107 obtains driving force by being in contact with this friction resistive body 614. Note that a higher friction coefficient of this friction resistive body 614 is not necessarily better. If the friction coefficient is too low, frictional resistance force (gripping force) resulting from the pressing force generated by the springs 603 and 613 on both sides in consideration of the friction coefficient does not output force to overcome rotational resistance or frictional resistance of the other driven shafts or the wiping cloth, which ends up idling with the thin film. On the other hand, if the friction coefficient of the friction resistive body 614 is too high, when a person forcibly holds down the thin film loop by a hand during its rotation, its resistance blocks the rotation of the friction resistive body 614, and as a result, also blocks the rotation of the motor 605, which becomes a cause of a damage due to motor overheating.

In this manner, setting values of the frictional force of the friction resistive body 614 and the springs 603 and 613 need appropriate setting ranges. This can be set by experimental means although there is a balance with other friction elements (resistances of the driven shafts, the wiping cloth, and the like).

Note that in FIG. 6, the friction resistive body 614 is a group of five divided hollow cylindrical parts, but this is due to a convenience in manufacturing of a prototype. The friction resistive body 614 may of course be a single hollow cylindrical part, or may also be simply a tape-shaped part having an appropriate friction coefficient.

The description continues further. A sensor and wiping sheet fixing bar 615 constituted by round shafts in two levels at a center of FIG. 6 is crossed like a bridge. This fixing bar may be regularly fixed without being moved. A thin film position sensor 616 which reads a position of the thin film is fixed to the sensor and wiping sheet fixing bar 615. In addition, since the sensor and wiping sheet fixing bar 615 has a structure in the upper and lower two levels, by using its elastically in the up and down direction, the wiping portion 302 can be attached and detached by way of one-touch snap.

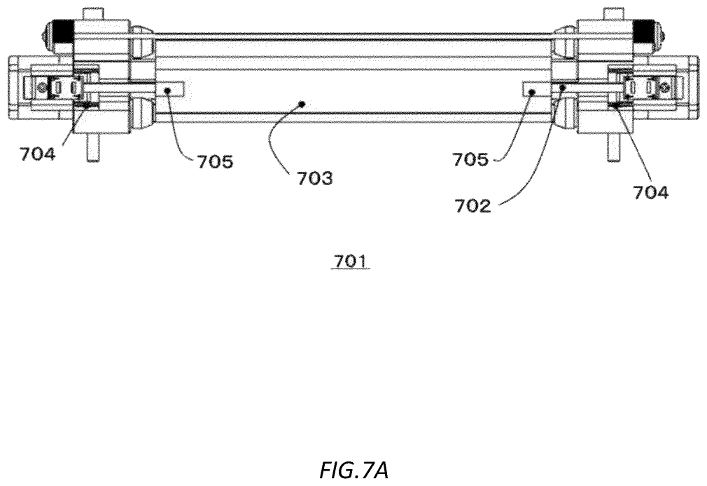

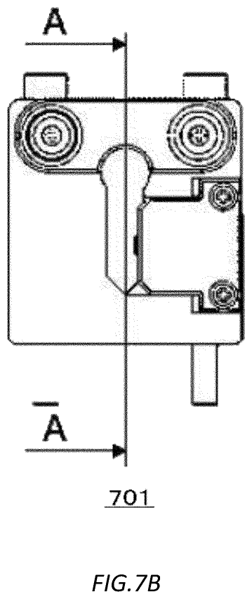

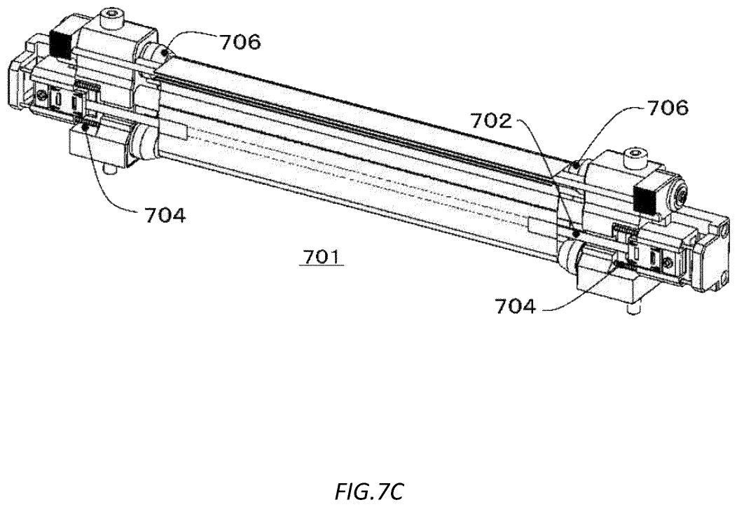

Next, a rotation drive portion 701 will be described by using FIG. 7A to FIG. 7C. FIG. 7B illustrates a cross-sectional view on one side, and FIG. 7C illustrates an isometric view. For a trial product, the SUS 304 foil is used as a heating disinfection band 702, and by causing a current to flow through the heating disinfection band 702, the heating disinfection band can be rapidly heated to a high temperature. The heated heating disinfection band 702 stretches out due to thermal expansion, and is not to be in contact with thin film 703 as it is. Thus, tension (preload) is regularly applied to the heating disinfection band from both sides by springs 704. The spring 704 is an example of a pulling portion which pulls the heating disinfection band 110 to compensate the stretch due to the heat of the heating disinfection band 110. Note that this spring 704 may be provided on only one side. This heating disinfection band 702 is connected, at a position out of a heating range, to a conductor (an aluminum foil or a copper foil) with a low resistance value (not illustrated). According to this structure, the high temperature heating can be applied to only the minimum necessary part. In addition, even when the heating disinfection band 110 and the thin film 703 are used which may be subjected to the thermal expansion by the heating, the sanitization apparatus 101 can disinfect the thin film 703 while the thin film is rotationally driven by pulling both the heating disinfection band 110 and the thin film 703. In addition, in order that the thin film loop is not to be shifted in a direction perpendicular to the travelling direction (that is, an apparatus longitudinal direction) during the travelling, multiple thin film traveling restriction pieces 706 are provided on both ends. By providing the thin film traveling restriction pieces 706, the shift of the thin film 107 in the direction orthogonal to the drive direction can be restricted.

The hardware of the sanitization apparatus has been described above. Next, software for effectively operating this sanitization apparatus will be described.

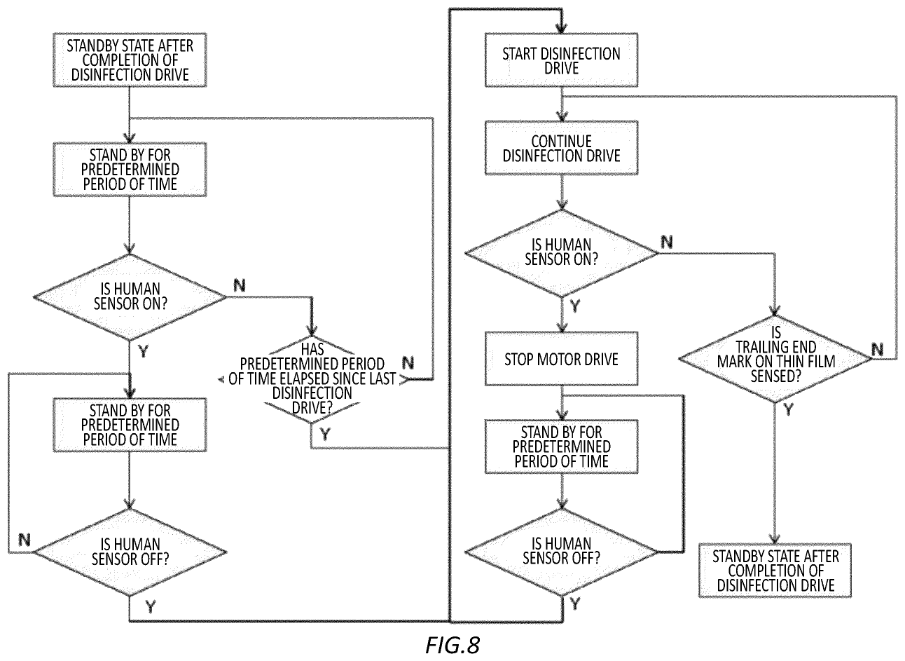

FIG. 8 illustrates a flowchart of a disinfection drive operation when a person uses the sanitization apparatus 101 from a standby state (which is a state in which the use is ready after the disinfection is completed) or when the use is not performed for a certain period of time. As illustrated in the drawing, such a flow is set that after a contact by a human hand is sensed by the human sensor in the standby state, when a release by the human hand is sensed, disinfection drive is started. In addition, such a flow is set that when the standby state continues for a long period of time, the disinfection drive is performed also after an elapse of any predetermined period of time. During the disinfection drive, a principle of the drive is that a human hand is not in contact, but when a human hand is in contact during the drive, the drive of the motor and the heating band is immediately stopped, so that safety of the person and the sanitization apparatus 101 is protected, and also the use by the person is prioritized. After the use by the person is ended, that is, after the human sensor turns OFF, the disinfection drive is started again. Note that at this time, when the last disinfection drive has not been completely rotated for a full thin film loop, the rotation is to be performed by being added with that much extra (not illustrated). In addition, an emergency stop during the disinfection drive may be performed not only by the sensing by the human sensor but also by the operational mismatching or timeout of the thin film encoder or the operational mismatching or timeout of the motor encoder (not illustrated).

In accordance with the sanitization apparatus as described above, it is possible to quickly disinfect and/or inactivate the grip surface to which the pathogen is adhered.

Note that although not illustrated in the drawing, the disinfection apparatus of the thin film does not necessarily need to be a heating body. For example, a similar disinfection effect may be attained by placing a cloth soaked in disinfectant (such as alcohol or a sodium hypochlorite solution) on a front side of the thin film at a position where the heating band is placed, and performing the travel such that the cloth is in contact with the thin film.

### INDUSTRIAL APPLICABILITY

The present invention can be widely used for surfaces of structural bodies that regularly require cleanliness. As an example of general use, the present invention is used as a cleaning apparatus for a product which is repeatedly contacted by an unspecified number of people in a short period of time, such as a doorknob, a gripping portion of a door, a handrail, or a hanging strap.

The present invention is also beneficial as usage for a medical instrument. In a general ward, it can be used for a handle of a door, a handrail of a bed, and the like in a hospital room to prevent hospital-acquired infections. It can also be used for a part where a hand is put on in a restroom. In addition, the present invention can be used for a structural body that is to be touched or grabbed in a state in which a person puts on gloves which require cleanliness during a surgery. The structural body is, for example, a grip portion or the like of a shadowless lamp in a surgery.

In this manner, possibilities for the industrial applications are wide ranging.

What is claimed is:

1. A sanitization apparatus comprising:
a rotation drive portion;
an annular thin film which has a predetermined perimeter and covers an outer circumference of the rotation drive portion; and
a fixed base portion to which the rotation drive portion is attached, wherein
the rotation drive portion has a disinfection portion which disinfects the thin film, and
the fixed base portion has a drive shaft which causes the thin film to perform rotational movement along the outer circumference of the rotation drive portion to cause the thin film to sequentially pass through the disinfection portion.

2. The sanitization apparatus according to claim 1, wherein
after the thin film is attached to the outer circumference, the rotation drive portion is attached to the fixed base portion.

3. The sanitization apparatus according to claim 1, wherein
the rotation drive portion has an opening and closing structure which opens and closes such that a perimeter of the rotation drive portion varies.

4. The sanitization apparatus according to claim 3, wherein
in a state in which the opening and closing structure is closed, the perimeter of the thin film is longer than a perimeter of a flange of the rotation drive portion.

5. The sanitization apparatus according to claim 3, wherein
in a state in which the opening and closing structure is opened, the perimeter of the thin film is longer than a perimeter of an outer circumference of the rotation drive portion which is covered by the thin film.

6. The sanitization apparatus according to claim 3, wherein
in a state in which the opening and closing structure is closed, the drive shaft causes the thin film to perform the rotational movement, and the disinfection portion disinfects the thin film.

7. The sanitization apparatus according to claim 1, wherein
the thin film has a thin film position mark for detecting a position of the thin film, and
the sanitization apparatus comprises a thin film position sensor which senses the thin film position mark to sense the rotational movement of the thin film.

8. The sanitization apparatus according to claim 1, wherein
the disinfection portion has a heating disinfection band which heats the thin film.

9. The sanitization apparatus according to claim 8, wherein
the heating disinfection band heats the thin film to 100 degrees Celsius or above.

10. The sanitization apparatus according to claim 8, comprising:
a pulling portion which pulls the heating disinfection band to compensate a stretch due to heat of the heating disinfection band.

11. The sanitization apparatus according to claim 1, comprising:
a wiping portion which removes a water droplet or dirt adhered to a surface of the thin film.

12. The sanitization apparatus according to claim 1, wherein
the fixed base portion has a position control portion which controls a position of the drive shaft to apply tension to the thin film.

13. The sanitization apparatus according to claim 10, wherein
the fixed base portion has a position control portion which controls a position of the drive shaft to apply tension to the thin film.

14. The sanitization apparatus according to claim 1, comprising:
a human sensor which senses use of the sanitization apparatus.

15. The sanitization apparatus according to claim 14, wherein
when the human sensor senses a person during the rotational movement of the thin film, the rotational movement of the thin film is stopped.

16. The sanitization apparatus according to claim 14, wherein
when the human sensor does not sense a person for a predetermined period of time, disinfection drive of the thin film is intermittently performed.

17. The sanitization apparatus according to claim 1, wherein
the drive shaft has a friction resistive body which causes the thin film to perform the rotational movement by frictional force against the thin film.

18. The sanitization apparatus according to claim 1, comprising:
a thin film traveling restriction piece which restricts shift of the thin film in a direction orthogonal to a drive direction.

19. The sanitization apparatus according to claim 1, wherein
a thickness of the thin film is 20 μm or less.

20. A doorknob comprising the sanitization apparatus according to claim 1.
